(19) United States
(12) Patent Application Publication
(10) Pub. No.: US 2010/0217201 A1
(43) Pub. Date: Aug. 26, 2010

(54) DEVICE FOR PROTECTING AND SECURING MEDICAL DEVICE AT A VENIPUNCTURE SITE

(75) Inventors: Chooi Tian Lee, Melaka (MY); Jan Kow Chan, Selangor (MY)

Correspondence Address: MAIER & MAIER, PLLC, 1000 DUKE STREET, ALEXANDRIA, VA, 22314, US

(73) Assignee: MEDIC DOMAIN (M) SDN. BHD., Melaka (MY)

(21) Appl. No.: 12/518,451

(22) PCT Filed: Nov. 29, 2007
(86) PCT No.: PCT/MY2007/000085

§ 371 Date: Jun. 10, 2009

(30) Foreign Application Priority Data
Jun. 19, 2008 (MY) PI20064648

(51) Int. Cl. A61M 39/00

(52) U.S. Cl. 604/177

(57) ABSTRACT

A device for protecting and securing at least a medical device at a venipuncture site and having at least a supporting member in which the medical tube winds around to form a loop, and having restrictor members to retain the loop formed around an arch-shaped passageway. The device is pivotally movable between an open position wherein the venipuncture site is exposed and a close position wherein the venipuncture site is covered and protected. The device further includes a fastening member to secure the device to the patient.

### RELATED APPLICATIONS

The present invention claims priority under 35 U.S.C. §120 to international patent application No. PCT/MY2007/000085, filed Nov. 29, 2007, which claims priority under 35 U.S.C. §119 to Malaysian Patent Application No. PI200664648, filed Dec. 11, 2006, the disclosures of which are incorporated by reference herein in their entireties.

### FIELD OF INVENTION

The invention relates to a device for protecting and securing medical device at a venipuncture site. In particular, it relates to a device for protecting and securing a venipuncture site so as to prevent any inadvertent removal of a catheter or IV needle from a patient, and to prevent any obstruction in fluid path in medical tube arising from any sudden tension.

### BACKGROUND OF INVENTION

A common practice in the treatment of patients involves utilizing a catheter or IV needle either to withdraw blood from patients or to introduce fluids and medication to a patient. It is thus necessary to stabilize the position of the catheter and to ensure that the catheter or IV needle is prevented from any inadvertent withdrawal.

When a catheter or IV needle is installed onto a patient, it is a general practice to retain the IV needle/catheter in subcutaneous position with an adhesive membrane or a tape. This simple method, however, does not offer sufficient protection against any inadvertent dislodgement of the catheter that might arise from unconscious and sudden movement. Patients, especially pediatric and geriatric, are likely to scratch or rub at the IV insertion site and consequently cause pain and potential risk of infection at the venipuncture site. There is thus a need to provide a means to both protect and secure the IV needle or catheter at the venipuncture site. Some of the known prior arts which aim to overcome the aforementioned problem have been disclosed in U.S. Pat. No. 6,540,721, U.S. Pat. No. 5,238,010, U.S. Pat. No. 5,116,324, U.S. Pat. No. 5,018,534 and U.S. Pat. No. 4,516,968. These devices, however, were only invented to cover or shield the venipuncture site and do not disclose any retaining means to prevent inadvertent dislodgement of IV needle/catheter or tube.

Other devices invented to anchor the IV needle/catheter and infusion line and to prevent the displacement of the IV needle/catheter from the subcutaneous condition have been disclosed in U.S. Pat. No. 6,447,485, U.S. Pat. No. 4,449,975, U.S. Pat. No. 4,059,105, U.S. Pat. No. 3,059,645, and U.S. Pat. No. 2,449,882. These devices, however, solely secure the catheter or IV needle, but do not provide the means to cover and protect the venipuncture site.

Furthermore, other devices invented to protect the venipuncture site as well as to anchor the catheter or IV needle from any inadvertent removal or withdrawal have been disclosed in U.S. Pat. No. 6,582,403, U.S. Pat. No. 4,846,807, U.S. Pat. No. 4,517,971, U.S. Pat. No. 4,397,647 and U.S. Pat. No. 3,900,026. However, although these devices could provide a solution to prevent the inadvertent withdrawal of IV needle or catheter, they have been known to induce kinking or crimping of medical tube which is undesirable as it obstructs the fluid path in the medical tube. One device which was invented to overcome the kinking problem is disclosed in U.S. Pat. No. 4,517,971.

The device covered in U.S. Pat. No. 4,517,971 utilizes a vein-bridge method by winding the medical tube around two pairs of cylindrical spools and studs. However, when the diameters of the cylindrical spools and studs are too small, kinking of the medical tube still occurs. On the other hand, if the diameters of the cylindrical spools and studs are too large, the device is not only cumbersome but also allows the medical tube to slide along the spools and studs rendering the device ineffective to secure the medical tube.

### SUMMARY OF INVENTION

It is an object of the present invention to provide a device for protecting and securing at least a medical device at a venipuncture site to prevent kinking in medical tube.

It is further object of the invention to provide a device for protecting and securing at least a medical device at a venipuncture site which prevents kinking in medical tube, and having adhesive or friction enhancement means to reduce excessive slippage movement of the device on a patient.

It is another further object of the invention to provide a device to secure either thick or thin medical tube while preventing kinking in the tube.

The present device aims to solve or substantially alleviate the difficulties encountered with the devices of the prior art.

The present device covers and protects a venipuncture site, and secures an IV needle or catheter from any inadvertent dislodgement. The device is preferably constructed of a semi-soft flexible and clear material which retains its convex-shape.

The cover of the present device is pivot movable between open and closed positions, and a reinforcement latching strip releasably holds the cover in its closed position.

Restrictor members of the present device hold or grip the medical tube to “immobilize” the portion of the medical tube near the catheter or IV needle.

A device according to a preferred embodiment of the invention will be apparent from the following detailed description, taken with reference to the accompanying drawings.

### BRIEF DESCRIPTION OF DRAWINGS

FIG. 1 is a bottom perspective of a device for protecting and securing venipuncture site in accordance with the first embodiment of the present invention.

FIG. 2 is a front perspective of the first embodiment with a strip member in closed position.

FIG. 3 is a top perspective view of the preferred latching strip member.

FIG. 4 is a top perspective of the first embodiment in a closed position having restrictor means for thin tube attached to a patient.

FIG. 5 is a top view of the first embodiment with adhesive members to attach the device to the patient.

FIG. 6 is a top view of the first embodiment having adhesive fastening patch and a releasable strap to attach the device to the patient.

FIG. 7 is a top view of a device for protecting and securing venipuncture site in accordance with the second embodiment of the invention.

FIG. 8 illustrate the top perspective views of the first embodiment when attached to the patient.

FIG. 9 illustrate the top perspective views of the second embodiment when attached to the patient.

### DETAILED DESCRIPTION OF PREFERRED EMBODIMENTS

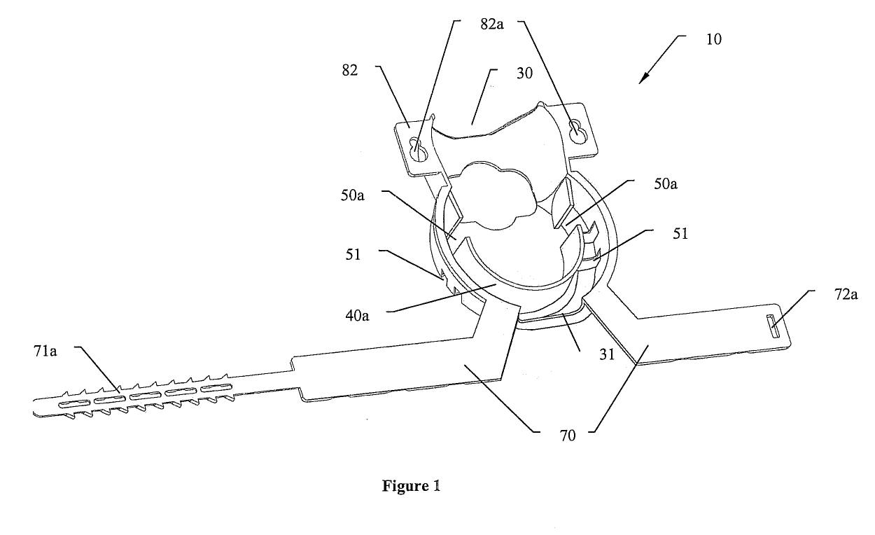

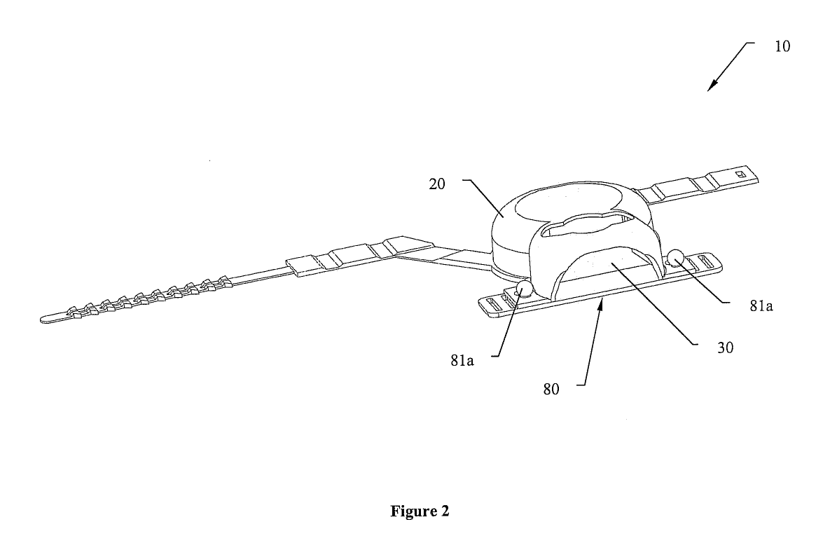

With reference to the drawings, and FIGS. 1 and 2 in particular, there is shown a device for protecting and securing at least a medical device at a venipuncture site (hereinafter referred to as “the present embodiment”) generally referred to by the numeral (10).

The present embodiment (10) is preferably constructed of a semi flexible translucent material which retains its shape. The present embodiment (10) comprises a convex-shaped cover (20) having a hollow interior. The cover (20) has at least an opening (30) to receive a medical tube (90), and an arch-shaped opening (31) at the other end which serves to avoid putting undue pressure on veins or on part of the medical device which is subcutaneously-located. The cover (20) is preferably pivotably movable between open and closed positions.

The cover (20) is integrally provided with a supporting member (40a), preferably in the form of an arch-shaped elongated wall within the cover, positioned near the opposite end of the cover's opening (30). The supporting member (40a) is arch-shaped to define a passageway (41) for the medical tube (90) to form a loop and ensures sufficient radial length is achieved. To retain the loop, a pair of restrictor members (50a) is provided—one is positioned at each end of the supporting member (40a)—to immobilize the tube. Once positioned around the supporting member (40a) and secured by the restrictor members (50a), the tube will not be able to slide freely on the supporting member (40a). Accordingly, the arch-shaped passageway serves to provide sufficient radial length of the medical tube (90) to allow for the restitution of the medical tube (90), upon release of application of stretch tensile force on the medical tube, resulting in the resilient medical tube regaining its proper shape, and maintaining an unobstructed flow of fluid in the medical tube.

The most preferred restrictor members (50a) are in the form of slots provided at the end portion of the arch-shaped elongated wall of the supporting member (40a). Other forms of restrictor members could also be used to effectively retain the tube on the supporting member (40a) or the loop formed.

The arch-shape of the supporting member, in combination with the restrictor members (50a) are desired to the working of the present embodiment (10). The supporting member (40a) and restrictor members (50a) could be embedded or integral with the cover. This assembly ensures that adequate radial length of medical tube (90) is provided thus allowing the tube to immediately return to its “relaxed” state whenever the force causing the tube to be flattened is removed. Additionally, the restrictor members (50a) ensure that the tube is retained on the supporting member (40a)—thus the assembly is maintained to prevent the tube from crimping and causing blockage in the medical tube. Accordingly, said restrictor members (50a) enable the medical tube securely retained on the supporting members (40a) to prevent any inadvertent of retraction of the medical tube, wherein the medical tubing is wound through the restrictor slots or hole took a curvature shape, when a sudden force is applied to the tubing, the applied force tends to straighten the tubing causing the tubing pressing on the edges of the slot or hole, resulting in the restriction in the movement of the tubing.

FIGS. 1 and 2 further disclose a preferred fastening means (70) in the form of a strap to engage the device to a patient's hand. To enable the strap to be fastened to a patient's hand, the preferred strap comprises a releasable knotted cord (71a) and locking apertures (72a).

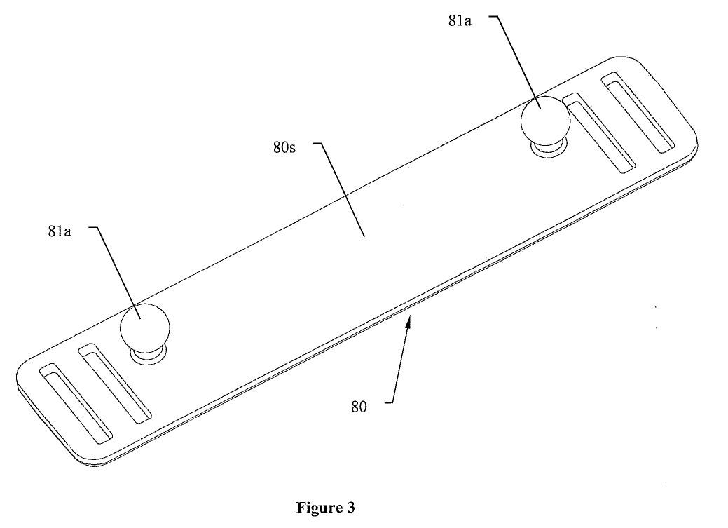

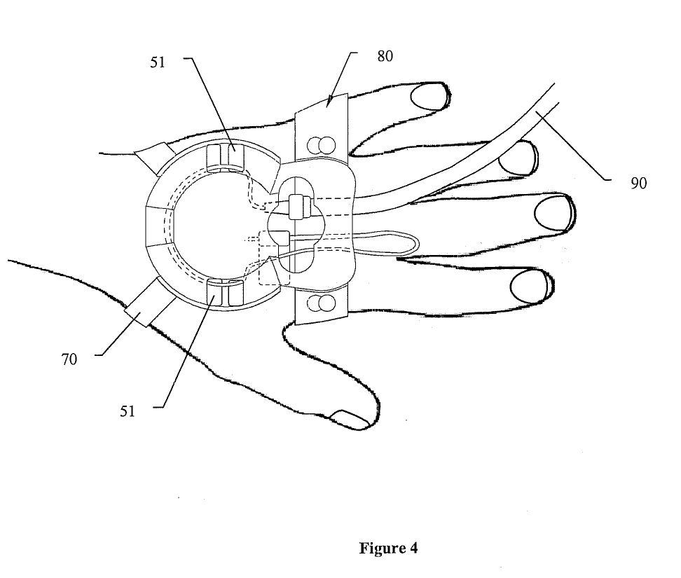

FIGS. 1, 2 and 3 disclose another fastening means (80) to stabilize the convex-shaped cover (20) when it is in the closed position. Said fastening means (80) is preferably a strip member (80s) attachable to a latching means (82) disposed at the cover (20). FIG. 3 discloses the strip member (80s) comprising at least a pair of male latching members (81a) in the form of studs integral with the strip member (80s) to be releasably interlockable with the female latching members (82a) provided on the cover (20). FIG. 4 discloses additional restrictor members (51), preferably in the form of a hole, to restraint thin medical tube from any stretch force. It is possible to weave the medical tube through those restrictor members and wind said medical tube around the elongated arch-shaped supporting strip to secure said tube. Additional mounting member may be provided at the strip member (80s) to support a portion of the medical tube. Other mounting members can also be provided on the strip member (80s) for use with other medical equipments.

Having noted the essential functions required, it is important to note that many design variations could be applied to the present assembly to obtain the required functions and effects.

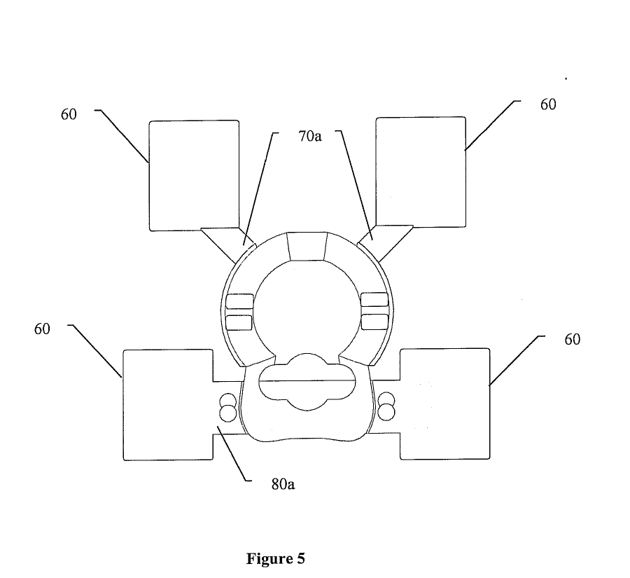

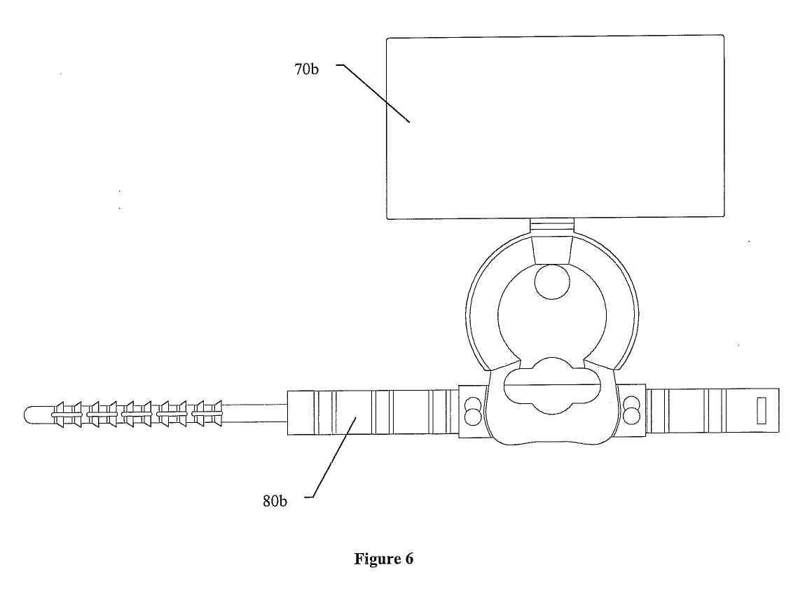

FIG. 5 and FIG. 6 disclose different embodiments of fastening means to attach the device to the patient. FIG. 5 discloses the use of adhesive means (60) for example adhesive pad secured to the fastening means (70a, 80a) respectively. FIG. 6 discloses the combination of different fastening means such as releasable fastening strap (80b) with an adhesive fastening patch (70b) to attach the device to the patient. Said fastening embodiments can have an anti-slippage means in form of sticky or friction-enhanced rubberized contact with the patients.

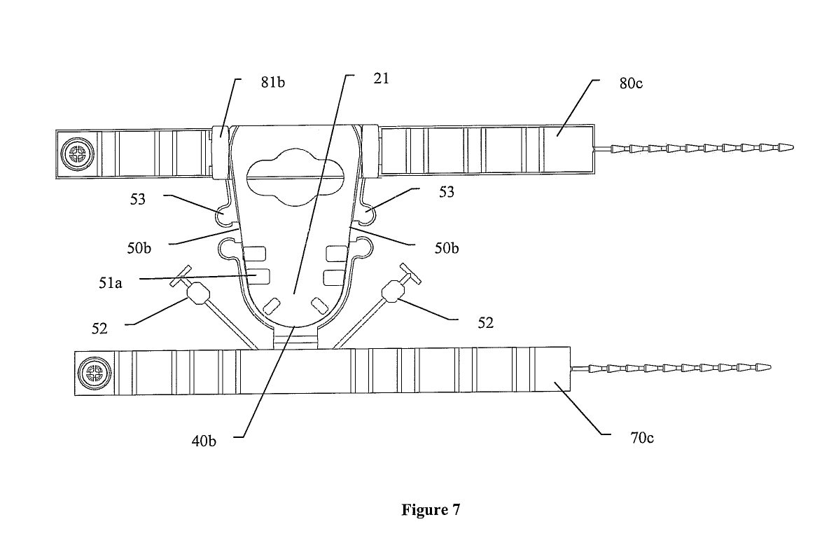

FIG. 7 discloses an alternative embodiment of the present invention wherein an arch-shaped passageway or supporting members (40b) is defined by the exterior of the convex-shaped structure of the cover (21) instead of the elongated arch-shaped wall as the supporting member. Instead of an elongated arch-shaped wall, it will be possible to wind the medical tube (90) around the convex-shaped cover (21). The medical tube (90) secure the loop formed when a pair of restrictor members (50b) in the form of slots coupled with tabs (53) along with an added pair of latching members (52) to secure the loop. Accordingly, the arch-shaped passageway serves to provide sufficient radial length of the medical tube (90) to allow the medical tube (90) upon release of application of stretch tensile force on the medical tube, resulting in the resilient medical tube regaining its proper shape, and maintaining an unobstructed flow of fluid in the medical tube.

As noted, the second assembly meets the gist of the present invention by providing the arch-shaped passageway allowing the medical tube (90) to form a loop on the cover (instead of within the hollow interior of the cover) and the restrictor members (50b) (in the form of a combination of slots and latching members (52)) to secure the loop. Said restrictor members (50b) enable the medical tube securely retained on the supporting members (40b) to prevent any inadvertent of retraction of the medical tube, wherein the medical tubing is wound through the restrictor slots or hole took a curvature shape, when a sudden force is applied to the tubing, the applied force tends to straighten the tubing causing the tubing pressing on the edges of the slot or hole, resulting in the restriction in the movement of the tubing.

The cover (21) also preferably includes further restrictor means (51a) to secure thin tube. Also provided is an alternative design of the fastening means (70c, 80c) to stabilize the device when it is in the closed position. However, different embodiments of fastening means to attach the device to the patient may be used. For example, adhesive pads or strips and the like, or combination of different fastening means such as releasable fastening strap with adhesive to attached the device to the patient may be used (not shown). The fastening embodiments can have an anti-slippage means in form of sticky or friction-enhanced rubberized contact with the patient.

The fastening means (80i c) disposed at the convex-shaped cover (21) may also be a strip member attachable to a latching means at the cover as described in the first embodiment of present invention. Said strip member may comprises at least a pair of male latching members in form of clip-on means (81b) integral with strip to be releasably interlocked with female latching members in the form of undercut or indentation provided on the cover (21) (not shown).

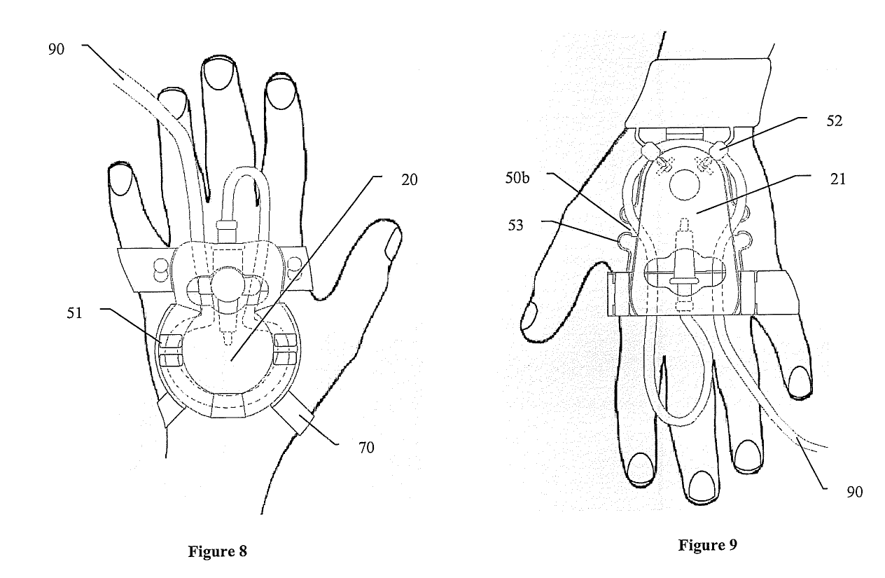

FIGS. 8 and 9 disclose the aforementioned two different embodiments when in use on a patient. As shown therein, the medical practitioner has a choice of applying either the first or second preferred embodiment to protect and secure a patient's venipuncture site.

In addition, the embodiments can incorporate further attachment means to enable attaching of other medical devices, such as a device to monitor bio-signals from the patient. Furthermore, both embodiments could incorporate means which reduce slippage or excessive lateral movement of the embodiments on the patient. This can be achieved by the embodiment having sticky or friction-enhanced rubberized contacts with the patients.

The invention may be embodied in other forms without departing from the scope or spirit of essential characteristics thereof. The present embodiments are therefore considered in all respects as illustrative and not restrictive, the scope of invention being indicated by the appended claims rather than by the foregoing description, and all changes which come within the meaning and range of equivalency of the claims are therefore intended to be embraced therein.

1. A device for protecting and securing at least a medical device at a venipuncture site comprising:
a cover with a hollow interior, said cover comprising an opening at one end to receive a portion of medical tube and an arch-shaped opening at other opposite end, the cover being pivotally movable between an open position wherein the venipuncture site is exposed and a closed position wherein the venipuncture site is covered and protected;
at least one supporting member embedded in the interior of said cover,
said supporting member defining an arch-shaped passageway along which the medical tube winds around internally to form a loop in the cover;
at least one restrictor member communicating effectively with the supporting member to retain the loop formed around the arch-shaped passageway; and
at least one fastening means disposed at pivotal end of the cover to secure the device to the patient;
wherein the cover serves to avoid putting undue pressure on venipuncture site or on part of the medical device that is subcutaneously-located;
wherein the arch-shaped passageway serves to provide sufficient radial length of the medical tube allow for the restitution of the medical tube, upon release of application of stretch tensile force on the medical tube, resulting in the resilient medical tube regaining its proper shape, and maintaining an unobstructed flow of fluid in the medical tube; and
said restrictor members enable the medical tube securely retained on the supporting members to prevent any inadvertent of retraction of the medical tube, wherein:
the tubing is wound through the restrictor slots or hole took a curvature shape, when a sudden force is applied to the tubing the applied force tends to straighten the tubing causing the tubing pressing on the edges of the slot or hole, resulting in the restriction in the movement of the tubing.

2. A device for protecting and securing at least a medical device at a venipuncture site claimed in claim 1 wherein said restrictor member is positioned at end portion of the supporting member, wherein said retaining restrictor members are slots which in association with the supporting member effectively accommodate and hold the medical tube (903-to prevent any inadvertent
dislodgement of IV needle or catheter from the patient.

3. A device for protecting and securing at least a medical device at a venipuncture site as claimed in claim 1 wherein the cover has further retaining comprises restrictor means to secure thin tube.

4. A device for protecting and securing at least a medical device at a venipuncture site as claimed in claim 1 wherein said cover further comprises has latching means comprising of at least a pair of female and/or male latching members to attach at least a fastening means with corresponding a male latching members and/or female latching members to stabilize the cover in the close position.

5. A device for protecting and securing at least a medical device at a venipuncture site as claimed in claim 4 wherein said fastening means is preferably a strip member having at least one pair of male and female latching members to interlock with at least a pair of corresponding female latching members and/or male latching members on the cover

6. A device for protecting and securing at least a medical device at a venipuncture site as claimed in claim 5 wherein the strip member further comprises additional fastening means to engage device to the patient.

7. A device for protecting and securing at least a medical device at a venipuncture site as claimed in claim 1, constructed of flexible material.

8. A device for protecting and securing at least a medical device at a venipuncture site as claimed in claim 1 wherein said device has anti-slippage means in form of sticky or friction-enhanced rubberized contacts with the patients.

9. A device for protecting and securing at least a medical device at a venipuncture site comprising:
a cover having at least an exterior supporting member being pivotally movable between open and closed positions, said supporting member defining an arch-shaped passageway by which the medical tube winds around to form a loop;
at least a restrictor member communicating effectively with the supporting member to retain the loop formed around the arch-shaped passageway; and
a fastening means disposed at the pivotal end of the cover to secure the device to a patient;
wherein the cover serves to avoid putting undue pressure on venipuncture site or on part of the medical device that is subcutaneously-located;
wherein the arch-shaped passageway serves to provide sufficient radial length of the medical tube to allow the medical tube upon release of application of stretch tensile force on the medical tube, resulting in the resilient medical tube regaining its proper shape, and maintaining an unobstructed flow of fluid in the medical tube; and
said restrictor members enable the medical tube securely retained on the supporting members to prevent any inadvertent of retraction of the medical tube, wherein:
the tubing is wound through the restrictor slots or hole took a curvature shape, when a sudden force is applied to the tubing, the applied force tends to straighten the tubing causing the tubing pressing on the edges of the slot or hole, resulting in the restriction in the movement of the tubing.

10. A device for protecting and securing at least a medical device at a venipuncture site as claimed in claim 9, wherein at least a slot on each side of said cover and said slots serve as restrictor members which effectively accommodate and hold the medical tube to prevent any inadvertent dislodgement of IV needle or catheter from the patient.

11. A device for protecting and securing at least a medical device at a venipuncture site as claimed in claim 9 wherein said cover has further comprises restrictor means to secure thin tube.

12. A device for protecting and securing at least a medical device at a venipuncture site as claimed in claim wherein said cover further comprises latching means with at least a pair of female and male latching members to attach at least a fastening means to stabilize the cover in the close position.

13. A device for protecting and securing at least a medical device at a venipuncture site as claimed in claim 12 wherein said fastening means is a strip having at least a pair of male latching members in form of clip-on means integral with strip to be releasably interlock with female latching members in the form of undercut or indentation provided at the cover.

14. A device for protecting and securing at least a medical device at a venipuncture site as claimed in claim 13 wherein the strip further comprises additional fastening means to engage device to the patient.

15. A device for protecting and securing at least a medical device at a venipuncture site as claimed in claim 9, constructed of flexible material.

16. A device for protecting and securing at least a medical device at a venipuncture site as claimed in claim 9 wherein said device has anti-slippage means in the form of sticky or friction-enhanced rubberized contacts with the patients.
